(19) United States
(12) Patent Application Publication
(10) Pub. No.: US 2005/0027269 A1
(43) Pub. Date: Feb. 3, 2005

(54) Male foreskin replacement for sensation preservation

(76) Inventor: Lee R. Brown, Old Bridge, NJ (US)

Correspondence Address: Charles I. Brodsky, Esq., 2 Bucks Lane, Marlboro, NJ, 07746, US

(21) Appl. No.: 10/629,037

(22) Filed: Jul. 30, 2003

(51) Int. Cl. A61F 13/15; A61F 2/50

(52) U.S. Cl. 604/378; 604/385.03; 623/66.1

(57) ABSTRACT

A male foreskin replacement for sensation preservation with anti-drip/anti-stain characteristic includes a multi-layer synthetic covering, an elasticized material to hold the covering in place encircling a user's penis when worn, and means actuable to open a closure path through the multi-layer fabric to a wearer's penis opening.

### CROSS-REFERENCE TO RELATED APPLICATIONS

NONE

### STATEMENT REGARDING FEDERALLY SPONSORED RESEARCH OR DEVELOPMENT

Research and development of this invention and Application have not been federally sponsored, and no rights are given under any Federal program.

### REFERENCE TO A MICROFICHE APPENDIX

NOT APPLICABLE

### BACKGROUND OF THE INVENTION

1. Field of the Invention

This invention relates to a male foreskin synthetic replacement, in general, and to one having the characteristics of preserving sensitivity while offering anti-urine drip/anti-urine stain protection at the same time, in particular.

2. Description of the Related Art

As is well known and understood, in past years, much research has been done on increasing male sexual sensitivity through the prescribing of medications and through the taking of various nutritional supplements. Although such takings have proven effective, they serve no purpose at all in preserving sensitivity adversely affected by such clothing as undershorts and trousers coming in contact with and/or rubbing against the male organ throughout the day. As will be understood, this becomes more recognizable once it is realized that some 90% of the male population in the United States undergo foreskin removal as a health protective measure, thereby removing the protection that is otherwise there provided.

### SUMMARY OF THE INVENTION

As will become clear from the following description, the present invention describes a synthetic replacement for the male foreskin which helps preserve sensation by allowing no clothing to ever touch the penis again—while ensuring that any such contact would only be had by the wearer or by the wearer's sexual partner. As will also be understood, the present invention could also serve in similar fashion to panty liners employed by females in providing an anti-urine drip/anti-urine stain characteristic by virtue of the covering provided—especially when worn by the older male population where a continuing urine flow after stoppage causes embarrassment.

As will be seen, the male foreskin replacement of the invention incorporates a multi-layer synthetic covering for the penis having four separate layers—a first in contact with the penis when worn, of a porosity to allow a urine stream to flow therethrough; a second overlying layer of characteristic to wick fluid away therefrom; a third layer overlying the second layer of a high fluid absorbency; and a fourth layer overlying the third layer porous to air and substantially impervious to fluid flow. An elasticized material is also provided, according to the invention, at one end of the fourth layer of the covering to encircle the penis in holding the multi-layer synthetic covering in place. Means are then provided on such fourth layer actuable on demand to open a closure path communicating through the four layers from a wearer's penis opening.

As will be appreciated, such covering can be worn through the day, with the openable closure actuated only when needed. Such elasticized material will be appreciated to hold the multi-layer covering in place, and can be in the nature of a band to be rolled some 1½″ onto the male organ, and just tight enough to be retained in position without unnecessary pressure.

In preferred embodiments of the invention, the first layer may be composed of a gauze-like material, sterile where desired, or of polypropylene. The third layer may be composed of a super absorbent polymer, such as one capable of holding up to eighty times its own weight in water. The fourth layer, serving as the outer protection against undershorts and trouser contact and irritation may be composed of a rubber, acetate, lambskin or polypropylene material. In any event, when it becomes time for the wearer to urinate, the openable closure is merely actuated open, and to be then secured after streaming has completed. As will be seen, such closure path opening may be actuated by a hook-and-loop adhesive arrangement, by an overlapping 2-component fold, or by a 2-component clasp arrangement, as an example.

### BRIEF DESCRIPTION OF THE DRAWINGS

These and other features of the present invention will be more clearly understood from a consideration of the following description, taken in connection with the accompanying drawings, in which:

FIG. 1 is a pictorial view of the male foreskin replacement of the invention for wearing in accordance with the illustration of FIG. 2;

FIG. 3 is an illustration of the foreskin replacement when opened; and

FIGS. 4-6 are illustrations helpful in understanding manners by which the foreskin replacement can be actuated open or closed in preserving sensitivity when worn.

### DETAILED DESCRIPTION OF THE INVENTION

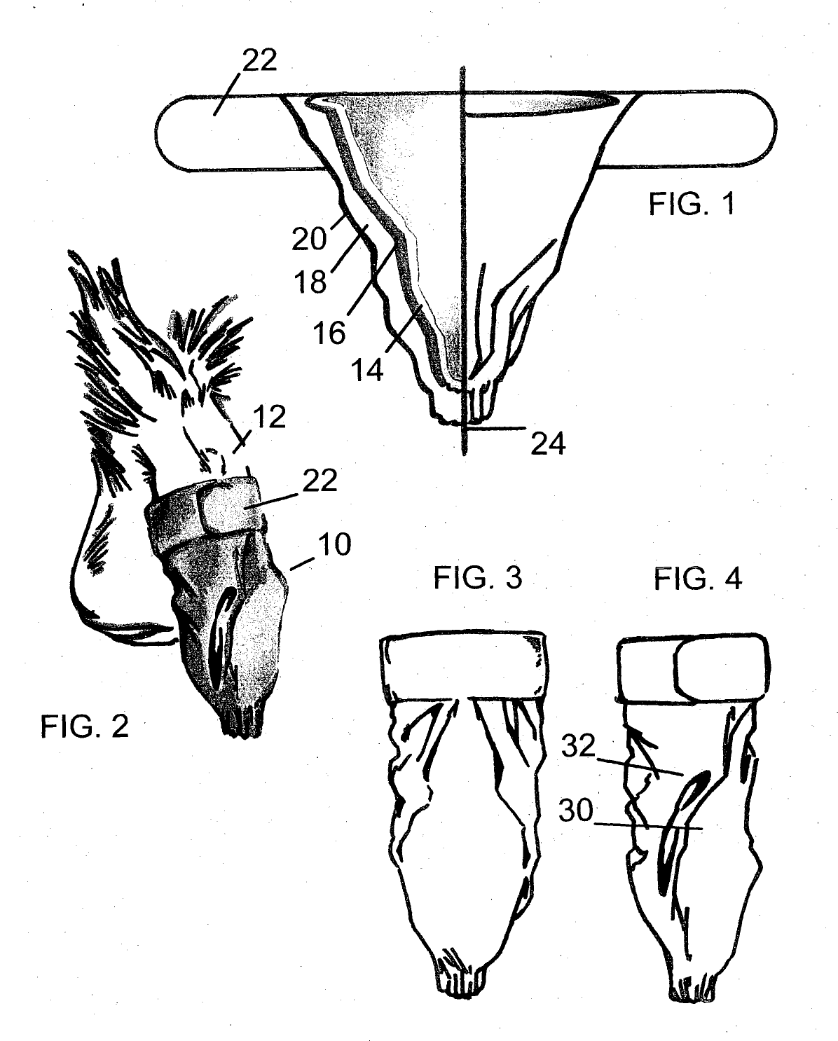

Referring to the drawings, the male foreskin replacement of the invention is shown as 10 in FIG. 2, to be worn on the male organ generally shown at 12. Such replacement unit 10 will be understood to be in the nature of a multi-layer synthetic covering having a first layer 14 (FIG. 1) in contact with the penis when worn, of a porosity to allow a urine stream to flow therethrough; a second layer 16 overlies the layer 14, and is of a characteristic able to wick fluid away from layer 14. In preferred embodiments of the invention, the layer 14 may be of a gauze-like material—sterile or otherwise—or may be of polypropylene plastic composition. A third layer 18 overlies the layer 16, and is selected of a high fluid absorbency, such as a super absorbent polymer, capable, for example, of holding up to eighty times its weight in water. A fourth layer 20 overlies the layer 18, and is porous to air while being substantially impervious to fluid flow. Such multi-layer synthetic will thus be seen to protect the male organ from contact with undershorts or trouser fabric when being worn during the course of a day. To hold the foreskin replacement 10 in place about the penis, an elasticized material 22 is included at one end of the layer 20 for encircling the organ to hold the multi-layer synthetic in position. Such elasticized material 22 may be of any conventional type, able to be rolled, for example, some 1½″ along the shaft of the penis. With the elasticized material band 22 rolled or otherwise closed as in FIG. 2, the foreskin replacement is able to be worn comfortably during the day, preserving the sensitivity of the organ, and protecting it from contact with any clothing worn, preserving it for the sanctity of the wearer's sexual partner.

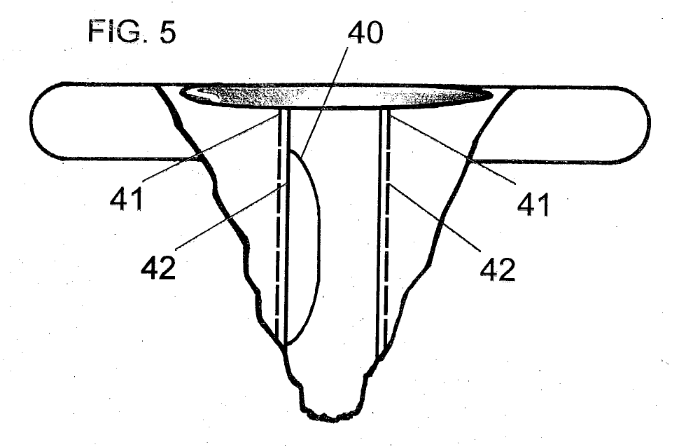

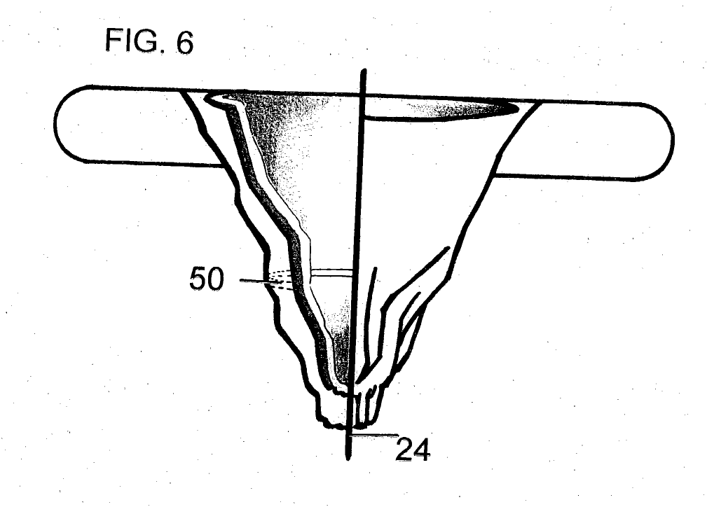

In further accordance with the invention, the foreskin replacement 10 further includes one of several available manners coupled with the layer 20 and actuable on demand, to open the closure path communicating through the layers to the penis opening of the wearer. With the closure path indicated along the axis 24, the direction of actuation to open the path leads to the view of FIG. 3, obtainable in one fashion through the use of an overlapping hook-and-loop adhesive combination shown as 30, 32 in FIG. 4. Where the opening of the closure path is controllable as by an overlapping 2-component fold of the kind typified in male undershorts, the openable closure would take on the appearance in FIG. 5 where the overlapping fold is illustrated by the reference notations 40 and 41-42. Any other type of openable closure could also be had, as with a button fly openable closure, a zip-locking type of closure or by a snap-fly type of closure along the lines of FIG. 6 with the closure control shown at 50.

As will be seen, the foreskin synthetic replacement of the invention can be selected of any type material as would protect the sensitivity of the male organ by keeping its skin thus insulated from any contacting fabric—thereby preserving its sensitivity for the wearer's partner. At the same time, it will be seen that the “porosity” characteristics and “wicking” abilities of the synthetic layers serve in absorbing any post urination fluid drip that might continue, in protecting the wearer's outer clothing against staining. Once the closure path of the synthetic is restored, the undershorts and trousers are thus protected, affording to the wearer the same type of panty liner protection as is affordable to the female of the species. In such manner, the foreskin replacement arrangement of the invention provides the dual purpose of sensitivity preservation and anti-drip/anti-staining.

While there have been described what are considered to be preferred embodiments of the present invention, it will be readily understood by those skilled in the art that modifications can be made without departing from the scope of the teachings herein. Thus, whereas a preferred embodiment of the invention provides quite adequate sensitivity preservation with the use of a fourth layer 20 composed of one of a rubber acetate, lambskin or polypropylene material, any type of material can be selected as long as it restricts a urine flow stream through it while providing sufficient insulation protection against clothing abrasion. For at least such reason, therefore, resort should be had to the claims appended hereto for a true understanding of the scope of the invention.

1. A male foreskin replacement for sensation preservation comprising:
a multi-layer synthetic covering for the penis having:
a) a first layer in contact with the penis when worn, of a porosity to allow a urine stream to flow therethrough;
b) a second layer overlying said first layer to wick fluid away therefrom;
c) a third layer overlying said second layer of high fluid absorbency; and
d) a fourth layer overlying said third layer porous to air and substantially impervious to fluid flow;
an elasticized material at one end of said fourth layer to hold said multi-layer synthetic covering in place when worn; and
means on said fourth layer actuable to open a closure path communicating through said first, second, third and fourth layers.

2. The male foreskin replacement of claim 1 wherein said first layer is composed of a gauze-like material.

3. The male foreskin replacement of claim 1 wherein said first layer is composed of a sterile gauze-like material.

4. The male foreskin replacement of claim 1 wherein said first layer is composed of polypropylene.

5. The male foreskin replacement of claim 1 wherein said third layer is composed of a super absorbent polymer.

6. The male foreskin replacement of claim 1 wherein said third layer is composed of a super absorbent polymer capable of holding up to eighty times its weight in water.

7. The male foreskin replacement of claim 1 wherein said fourth layer is composed of one of rubber, acetate, lambskin and polypropylene.

8. The male foreskin replacement of claim 1 wherein said first layer is composed of polypropylene, wherein said third layer is composed of a super absorbent polymer capable of holding up to eighty times its weight in water, and wherein said fourth layer is composed of one of rubber, acetate, lambskin and polypropylene.
